(19) United States
(12) Patent Application Publication
(10) Pub. No.: US 2008/0306501 A1
(43) Pub. Date: Dec. 11, 2008

(54) Device and method for ultrasound wound debridement

(75) Inventor: Eilaz Babaev, Minnetonka, MN (US)

Correspondence Address: ROPES & GRAY LLP, PATENT DOCKETING 39/41, ONE INTERNATIONAL PLACE, BOSTON, MA, 02110-2624, US

(73) Assignee: Celleration, Inc., Eden Prairie, MN (US)

(21) Appl. No.: 12/228,331

(22) Filed: Aug. 12, 2008

Related U.S. Application Data
(63) Continuation of application No. 10/444,296, filed on May 23, 2003.
(60) Provisional application No. 60/401,685, filed on Aug. 7, 2002.

(51) Int. Cl. A61B 17/32

(52) U.S. Cl. 606/169

(57) ABSTRACT

An end-effector configured and dimensioned for being connected to an ultrasound surgical device is provided. The end-effector includes means for receiving ultrasound waves generated and propagated by the ultrasound surgical device, and means for debriding tissue on a wound surface. The means for debriding tissue includes at least one non-smooth surface; and means for propagating the received ultrasound waves to the at least one non-smooth surface for vibrating the at least one non-smooth surface, wherein the at least one non-smooth vibrating surface is configured to contact and debride tissue on the wound surface. A method is further provided for debriding wound tissue including the steps of providing ultrasound waves to a non-smooth surface configured for debriding, propagating the ultrasound waves to cause vibration of the non-smooth surface, and contacting the vibrating surface configured for debriding with the wound tissue.

### PRIORITY

This application claims priority to a U.S. Provisional Application filed on Aug. 7, 2002 and assigned U.S. Provisional Application Ser. No. 60/401,685, the contents of which are incorporated herein by reference in their entirety.

### BACKGROUND OF THE INVENTION

### Field of the Invention

This invention relates to a device and method for performing ultrasound medical procedures, and particularly to a device and method for performing ultrasound debridement of a wound.

### BACKGROUND OF THE INVENTION

The wound healing process is often complicated by the presence of non-viable, devitalized necrotic tissue in the wound area. A debridement procedure includes removal of the necrotic tissue to prevent infection and accelerate healing of the wound.

There has been a long-standing need for a new surgical device for debridement of wounds, which would significantly enhance wound healing. All mechanical, hydraulic/fluidic and other surgery debridement methods and devices, including contemporary ones such as laser or enzymatic debridement have significant disadvantages. For example, in mechanical debridement methods using surgical knives, scissors or other sharp-edged instruments, due to the high mechanical force required between the instrument and biological tissue tends to cause bleeding, inflict mechanical trauma, and hard to control depth of debris removal. Laser debridement of wounds is very slow, tends to injure the underlying tissue, and ranges from a “laser-vaporization” or “laser-excision” effect to a scalpel effect, based on temperature. An example of a laser debridement method is described in U.S. Pat. No. 5,342,352 to Franken.

Enzymatic debridement methods have disadvantages, such as formation of remnants of formulations, such as denatured proteins, on the wound surface, reduced granulation tissue, and potential occurrence of local or systemic toxicity. An example of enzymatic debridement is described in U.S. Pat. No. 5,145,681 to Fortney.

Ultrasonic surgical methods are described in U.S. Pat. No. 4,188,952 to Loschilov et which describes an ultrasonic knife with triangular shaped teeth for separation of cartilage tissue, U.S. Pat. No. 3,589,363 to Banko, U.S. Pat. No. 4,428,748 to Peyman et al., and U.S. Pat. No. 6,270,471 to Hechel et al., which describe the use of ultrasound for dissection of soft tissue cells together with irrigation, aspiration and emulsification procedures. U.S. Pat. No. 6,280,407 to Manna et al., describes an ultrasonic dissection and coagulation system.

The above prior art ultrasound surgical devices have a blade having either a straight or a serrated sharp edge, where teeth of the serrated edge are aligned along a single row forming a one-dimensional array. A forced applied to the blade for cutting is distributed over the surface area that contacts the tissue. The ultrasonic vibrations enhance the cutting power of the blade so that a substantial cut may be effected by applying only a small force to the blade. Due to the enhanced cutting power and distribution of the applied force over a relatively small contact surface area, control of the cutting depth is difficult to achieve. Furthermore, the prior art ultrasound surgical devices are not suitable for debridement procedures which are best performed with blade(s) having a large contact area.

Accordingly, it is an object of the present invention to provide a device and method for ultrasonic wound debridement.

It is another object of the present invention to treat wounds for the healing thereof using an ultrasound apparatus designed to contact the wound while performing simultaneous debridement of the wound.

It is a further object of the present invention to increase depth control of cutting performed by the ultrasound device using an increased contact area.

It is yet a further object of the present invention to provide a device and method for delivering or removing medical or non-medical fluids and materials to or from the wound area.

### SUMMARY OF THE INVENTION

In accordance with the present invention, an end-effector configured and dimensioned for being connected to an ultrasound surgical device is provided. The end-effector includes means for receiving ultrasound waves generated and propagated by the ultrasound surgical device, and means for debriding tissue on a wound surface. The means for debriding tissue includes at least one non-smooth surface; and means for propagating the received ultrasound waves to the at least one non-smooth surface for vibrating the at least one non-smooth surface, wherein the at least one non-smooth vibrating surface is configured to contact and debride tissue on the wound surface.

In a preferred embodiment, the at least one non-smooth surface includes at least one protrusion and at least one sharp edge. In a more preferred embodiment the at least one non-smooth surface includes a two-dimensional array of teeth. In preferred embodiments the means for propagating includes a channel at least partially circumscribed with an indentation for causing at least a portion of the ultrasound waves to be configured from the group consisting of torsional and rotational waves for causing at least a portion of the at least one non-smooth surface to rotate. Preferably, the end-effector is configured and dimensioned for directly propagating at least a portion of the ultrasound waves to the wound surface for sonication thereof and for providing a therapeutic effect to the wound.

In still another embodiment of the invention, a method is provided for debriding wound tissue. The method includes the steps of providing ultrasound waves to a non-smooth surface configured for debriding, propagating the ultrasound waves to cause vibration of the non-smooth surface, and contacting the vibrating surface configured for debriding with the wound tissue.

### BRIEF DESCRIPTION OF THE DRAWINGS

Various embodiments of the invention will be described herein below with reference to the figures wherein:

FIG. 1 is a perspective view of an ultrasound wound debridement system in accordance with the present invention;

FIG. 2 is perspective view of an ultrasound tip for the ultrasound wound debridement system illustrated in FIG. 1;

FIGS. 3A-3J are perspective views of a variety of tooth configurations for the ultrasound tip illustrated in FIG. 2;

FIG. 4A is a schematic lateral cross-sectional view of the ultrasonic tip illustrated in FIG. 2, with a flow channel for irrigation or aspiration configured in accordance with a first embodiment;

FIG. 4B is a schematic lateral cross-sectional view of the ultrasonic tip illustrated in FIG. 2, with a flow channel for irrigation aspiration configured in accordance with a second embodiment;

FIG. 4C is a schematic lateral cross-sectional view of the ultrasonic tip illustrated in FIG. 2, with a flow channel for irrigation or aspiration configured in accordance with a third embodiment;

FIG. 5A is a schematic lateral cross-sectional view an ultrasonic tip illustrated in FIG. 2 in accordance with another embodiment;

FIG. 5B is a schematic lateral cross-sectional view of the ultrasonic tip illustrated in FIG. 5A, with a flow channel for irrigation or aspiration;

FIG. 6A is a schematic view of an ultrasound transducer with tip in accordance with a preferred embodiment of the invention; and

FIG. 6B is a schematic view of the tip shown in FIG. 6A during a debridement procedure.

### DETAILED DESCRIPTION OF THE PREFERRED EMBODIMENT

The present invention provides a method and system for debriding and treating wounds, such as for use in cosmetic or general surgery. An ultrasound system is provided that creates, directs and delivers ultrasound waves to a wound surface and debrides while simultaneously treating the wound therapeutically with the ultrasound waves (sonication). Ultrasonic waves are directed to a sharp edge of an ultrasonic tip for the debridement and treatment of the wound. The ultrasonic system further provides for debridement of the wound with controllable depth as well as irrigation and suction (aspiration) of debris at the wound area, such as fragmented necrotic tissue, foreign matter, and/or contaminants. The ultrasonic system further energizes drugs for irrigation and causes penetration of the drug to below the surface of the wound. The ultrasound waves create acoustical and mechanical forces, which provide therapeutic effect and/or enhance direction of the drug and penetration of the drug below the surface of the wound.

With reference to FIG. 1, an ultrasound generator system 10 is shown. The ultrasound generator system 10 includes an ultrasound generator 12 having a front panel 14 having various regulatory controls for controlling power, display, intensity, etc. Ultrasound generator 12 is connected to an ultrasound transducer 16 by a cable 18. The ultrasound transducer 16 includes a tip 20 receiving ultrasound waves produced by the transducer 16. The tip 20 includes a specially designed distal end 22 with a wound debridement surface 30 configured for debriding. The wound debridement surface 30 is preferably a non-smooth surface. In a preferred embodiment, the wound debridement surface 30 includes at least one protrusion.

In another preferred embodiment, the at least one protrusion includes at least one tooth 32, preferably configured in an array 31, where preferably the array 31 is two-dimensional. Preferably, the teeth 32 have one or more edges or points that are preferably honed to razor sharpness for cutting and/or scraping, herein referred to as sharp edges.

Longitudinal ultrasound waves traveling in the direction shown by arrow 34 cause the edges 32 to debride the wound and the ultrasound waves to sonicate the wound as the ultrasound transducer 16 is operated by a user applying a slight mechanical touch for gently contacting the transducer tip 20 to the surface of the wound. The user may further operate the ultrasound transducer 16 by applying motion in the direction shown by arrow 36. The ultrasound transducer 16 is shown formed of a sandwich transducer design, and may be formed of another design such as for an ultrasound therapy transducer, as is known in the art.

Ultrasound waves produced preferably have frequencies ranging from 10 kHz-10⁶ MHz, and preferably 20 kHz-100 kHz, which are capable of achieving amplitudes ranging from 1-300 microns, preferably 30-200 microns, more preferably 30-150 microns, and most preferably 30-100 microns. A working range in which amplitudes range from 5-30 microns is sufficient for many applications, however the higher amplitudes ranging from 30-300 microns provide benefits during debridement and/or treatment, as described further below. The lower frequencies are better able to achieve the higher amplitudes, and the desired amplitude depends upon the application, such as the condition of the wound being treated. The frequency may be modulated or pulsed. Ultrasound waves driving the ultrasonic tip have waveforms, such as sinusoidal, rectangular, trapezoidal, triangular and a combination thereof, where rectangular is the preferred waveform.

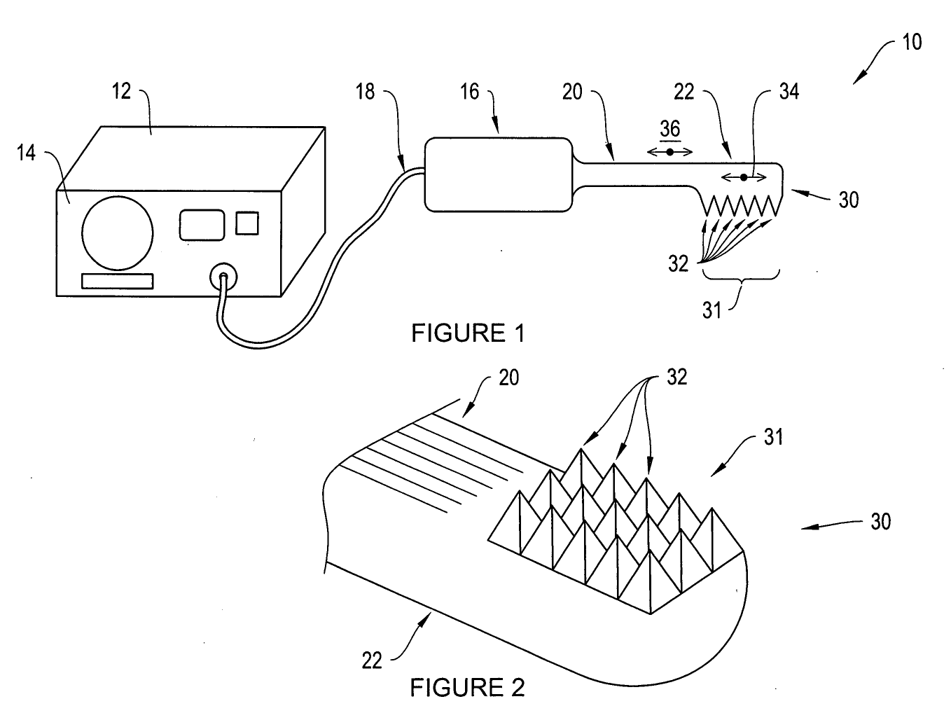

With reference FIG. 2, an exemplary two-dimensional array 31 is shown. The tip 20 includes a mounting surface, wherein the teeth 32 are mounted to the mounting surface at a proximal end of the respective teeth. Each tooth 32 includes a contact surface for making contact with surface of the wound during a debridement procedure. Preferably, the total contact area, including the combined contact surface of each tooth 32, has a substantial length and width. The total contact area is distributed across an area that corresponds approximately to the area dimensions of the two-dimensional array 31.

In conventional ultrasonic surgical devices, which are designed for procedures such as separating, breaking apart, cutting, dissecting, etc., tissue, the sharp edge is arranged in a one-dimensional configuration having a continuous sharp edge or a discontinuous sharp edge including a one-dimensional array of teeth. Accordingly, the total contact area of the sharp edge corresponds to the collective contact surface(s) of the one-dimensional sharp edge, and the total contact area is distributed across an area that corresponds approximately to the area dimensions of the one-dimensional array of teeth or the continuous sharp edge.

The two dimensional configuration of array 31 of the wound debridement surface 30 of the inventive ultrasound wound debridement system 10 provides an increased total contact surface area relative to an ultrasonic surgical device having a one-dimensional configuration of a sharp edge of similar length. The cutting force provided by the ultrasound energy and mechanical force applied by the user using the inventive system is distributed across the increased total contact surface area. The distribution of the multiple contact surfaces about the area occupied by S the two-dimensional array 31 causes further distribution of the cutting force. Distribution of the cutting force provides increased depth control and the capability to debride and sonicate, while preventing unwanted tissue penetration and unwanted bleeding. An alteration of the energy level of the ultrasound waves and/or the mechanical force provided to the array 31 is distributed among the teeth 32 of the array 31, allowing fine control of the vibrations of the individual teeth 32 and of the debridement process.

Use of ultrasound waves generated at low frequencies, such as 20 kHz-100 kHz and having high amplitudes, such as 30-300 microns promotes cavitation of the ultrasound waves and penetration of the ultrasonic waves below the surface of the wound to accelerate the healing process.

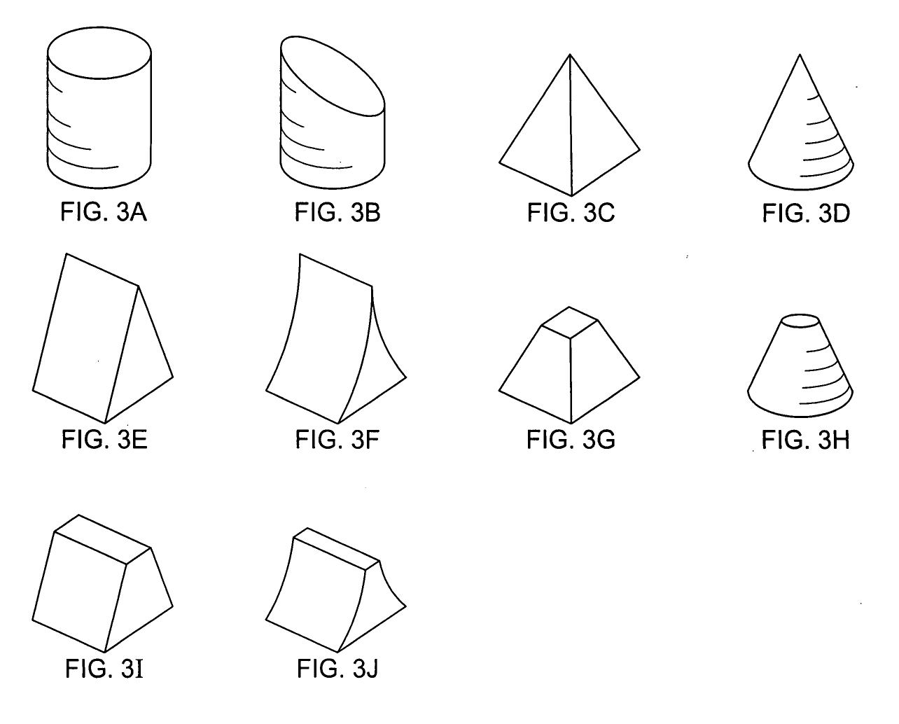

With reference to FIGS. 3A-3J, a variety of tooth configurations for teeth 32 are shown. The array 31 may include teeth 32 having one of the configurations shown or a combination thereof. Preferably, the individual teeth 32 are tapered so that the width of the tooth 32 is greater at a proximal end than at a distal end of the tooth for promoting greater resistance during a debridement procedure and controlling penetration. Tooth configurations shown include a cylinder, cylinder with angled cut, pyramid, cone, wedge, tapered wedge, truncated tapered wedge, etc.

Furthermore, selection of the shape and size of the transducer tip 20 in combination with selection of the frequency and amplitude of the ultrasonic energy used is advantageous in achieving the desired sonication effect and debridement effect.

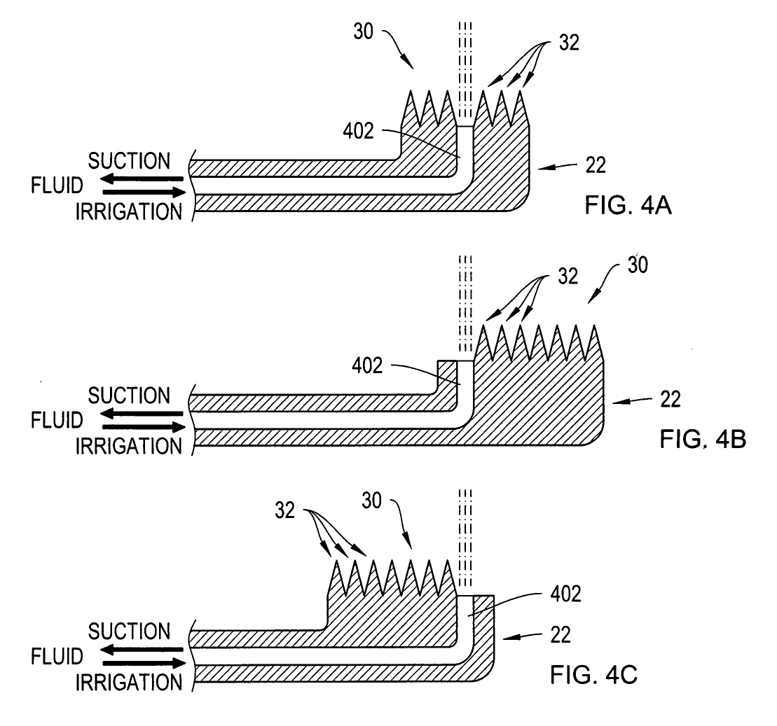

FIGS. 4A-4C show the ultrasound transducer tip 20 including a channel 402 for irrigation and/or suction, i.e. aspiration. The ultrasound transducer tip 20 is configured with a sandwich transducer design, however other transducer configurations may be used. The channel 402 is connected to a source of irrigation and/or suction. For irrigation, the source of irrigation provides a powder or fluid, such as saline, liquid medicine, antibiotics, etc., to the wound area. For suction, the severed and/or debridement tissue and/or foreign matter or contaminants are suctioned off from the wound through the channel 402. Preferably, suction and/or irritation are activated and/or controlled by the operator of the transducer 16 while performing a wound debridement procedure, and most preferably, with the same hand that operates the transducer 16.

In a preferred embodiment shown in FIG. 4A, the opening of channel 402 is located within the tip's wound debridement surface 30. In another embodiment shown in FIG. 4B, the opening of channel 402 is located at a proximal end of the tip's wound debridement surface 30. In another embodiment shown in FIG. 4C, the opening of channel 402 is located at a distal end of the tip's wound debridement surface 30. Separate channels may be provided for irrigation and suction, or one channel may be provided for selective alternate use for irrigation and suction. Irrigation and/or suction may be performed during a debridement procedure, or separate from a debridement procedure.

The shape and size of the transducer tip 20, the large contact area of the array provides for therapeutic use of the ultrasound energy, and/or the use of low frequencies, such as 20 kHz-100 kHz and high amplitudes, such as 30-300 microns, promote the formation of force and pressure of the ultraviolet waves and cavitation thereof, causing penetration of the ultrasound waves into the tissue, providing a therapeutic effect. When medicament, in the form of a fluid or a powder, is applied to the tissue the pressure formed and cavitation of the ultrasound waves causes penetration of the medicament into the tissue, providing a further therapeutic effect for accelerating the healing process.

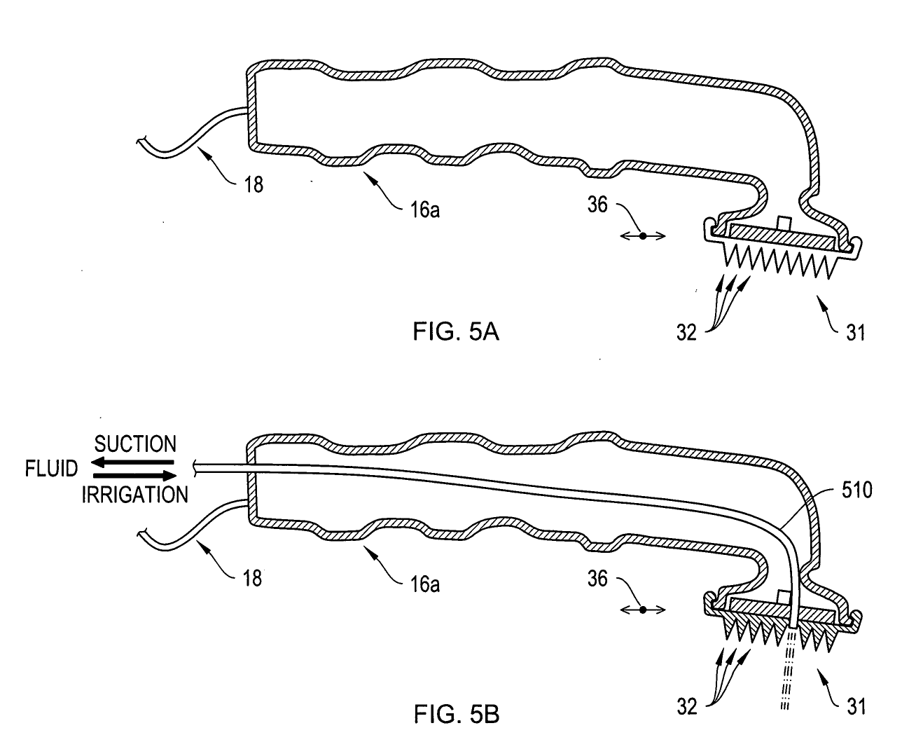

FIGS. 5A and 5B show an ultrasound transducer 16a in accordance with a further embodiment of the invention, where transducer 16a is similar to transducer 16 of FIG. 1, except that ultrasound transducer is configured as an ultrasound therapy transducer. Transducer 16a includes the two-dimensional array 31 of teeth 32 and cable 18, similar to transducer 16 of FIG. 1. In FIG. 5B, the ultrasound transducer 16a is shown to include a channel 510 for irrigation and/or suction purposes, similar to channel 402 of FIGS. 4A-4C.

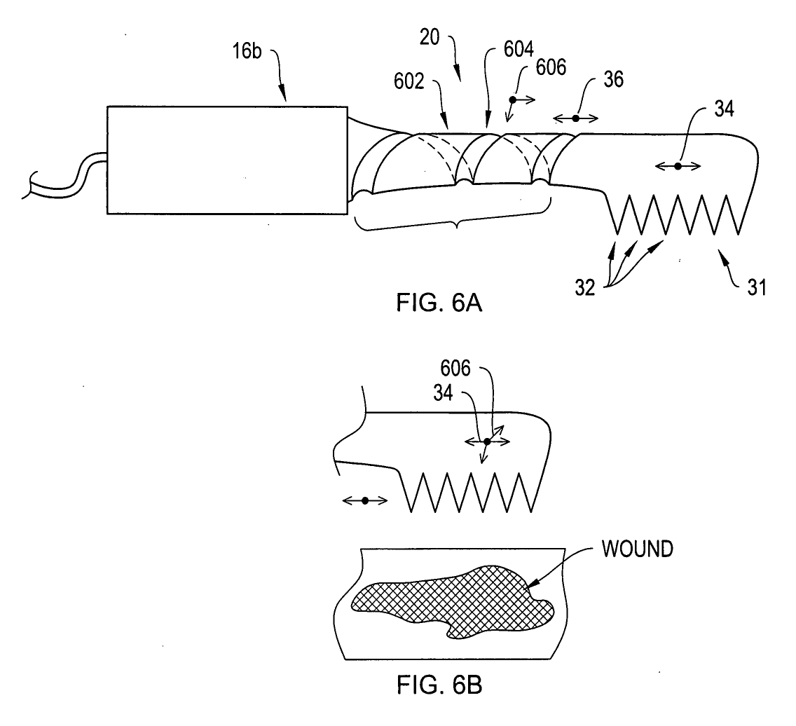

FIGS. 6A and 6B show an ultrasound transducer 16b in accordance with another embodiment of the invention Ultrasound transducer 16b, which is similar to transducer 16 of FIG. 1, includes a channel, such as neck portion 602, for propagating the ultrasound waves to the distal tip 22. The channel may be configured or shaped in accordance with the application. The channel is provided with at least one indentation that is configured to at least partially circumscribe the channel. The indentation may be, for example, a thread, slot, groove or combination thereof. Wall(s) of the indentation may include one or more walls, where the wall(s) may be straight and/or curved, and a cross-sectional shape of the indentation at a point along the length of the indentation may be a shape such as square, rectangular, trapezoidal, triangular, etc. In the embodiment shown, an indentation is provided in the configuration of a spiral, continuous groove 604 formed along at least a portion of the length of the neck portion 602. Transducer 16b is shown to include array 31 of teeth 32, similar to transducer 16. It is contemplated that the tip 20 may be alternatively configured with a one dimensional array of one or more teeth, one or more toothless blades, a flat contact surface area, etc.

The groove 654 creates torsional and rotational waves moving in the direction shown by arrows 606, which act upon the teeth 32, in addition to the longitudinal waves shown to be moving in the directions shown by arrows 34. The longitudinal waves act upon the teeth 32 to cause the teeth 32 to vibrate in a back and forth one-dimensional motion. The torsional and rotational waves act upon the teeth 32 to cause the teeth 32 to vibrate in a rotational motion having two-component movement for two-dimensional movement. The rotational vibration of the teeth 32 improves the effectiveness of the teeth 32 for performing debridement procedures. A user may further move the ultrasonic transducer 16b, such as in a direction as shown by the arrows 36. The rotational and torsional waves further act upon the tissue of the wound for providing a therapeutic effect.

It is contemplated that internal debridement may be performed using an ultrasound transducer 16, 16a or 16b configured for internal use.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. An end-effector configured and dimensioned for being connected to an ultrasound device, said end-effector comprising:
means for receiving ultrasound waves generated and propagated by the ultrasound device; and
means for debriding tissue on a wound surface and enhancing wound healing, said means for debriding tissue and enhancing wound healing comprising:
at least one non-smooth surface; and
means for propagating the received ultrasound waves to the at least one non-smooth surface for vibrating the at least one non-smooth surface, wherein the at least one non-smooth vibrating surface is configured to contact and debride tissue on the wound surface and to enhance wound healing.

2. The end-effector in accordance with claim 1, wherein the at least one non-smooth surface includes a contact area for contacting and debriding the wound surface, and wherein the contact area is arrayed in a radial or circular pattern.

3. The end-effector in accordance with claim 1, wherein the at least one non-smooth surface includes at least one protrusion.

4. The end-effector in accordance with claim 3, wherein the at least one protrusion includes at least one tooth.

5. The end-effector in accordance with claim 1, wherein the at least one non-smooth surface includes a two-dimensional array of teeth.

6. The end-effector in accordance with claim 1, wherein the at least one non-smooth surface includes a two-dimensional grid of teeth.

7. The end-effector in accordance with claim 1, wherein the at least one non-smooth surface includes a contact area for contacting and debriding the wound surface, the contact area having a predetermined length and width.

8. The end-effector in accordance with claim 1, wherein the at least one non-smooth surface includes a plurality of teeth, and wherein each of said plurality of teeth has the same tooth configuration.

9. The end-effector in accordance with claim 8, wherein the tooth configuration is selected from a cylinder or a cylinder with an angled cut.

10. The end-effector in accordance with claim 8, wherein the tooth configuration is a pyramid.

11. The end-effector in accordance with claim 8, wherein the tooth configuration is a cone.

12. The end-effector in accordance with claim 8, wherein the tooth configuration is selected from a wedge, a tapered wedge, or a truncated tapered wedge.

13. The end-effector in accordance with claim 1, wherein the means for propagating includes a channel at least partially circumscribed with an indentation for causing at least a portion of the ultrasound waves to be configured as torsional or rotational waves for causing at least a portion of the at least one non-smooth surface to rotate.

14. The end-effector in accordance with claim 1, wherein the ultrasound waves have frequencies in the range from 20 kHz-100 kHz.

15. The end-effector in accordance with claim 1, wherein the ultrasound waves have amplitudes in the range from 5 to 30 microns.

16. The end-effector in accordance with claim 1, wherein the ultrasound waves have amplitudes in the range from 1 to 100 microns.

17. The end-effector in accordance with claim 1, wherein the ultrasound waves have amplitudes in the range from 30-300 microns.

18. The end-effector in accordance with claim 1, wherein the ultrasound waves have amplitudes in the range from 30-120 microns.

19. The end-effector in accordance with claim 1, wherein at least one of the end-effector and the received ultrasound waves is configured and dimensioned for forming at least one of acoustical and mechanical pressure with the received ultrasonic waves for causing a therapeutic effect to the wound surface.

20. The end-effector in accordance with claim 1, further comprising at least one channel in fluid communication with an area adjacent the wound surface for transfer of material to or from the wound surface.

21. An end-effector configured and dimensioned for being connected to an ultrasound device, said end-effector comprising:
means for receiving ultrasound waves generated and propagated by the ultrasound device;
means for debriding tissue on a wound surface and enhancing wound healing, said means for debriding tissue and enhancing wound healing comprising:
at least one non-smooth surface, which non-smooth surface is arrayed in a radial or circular pattern; and
means for propagating the received ultrasound waves to the at least one non-smooth surface for vibrating the at least one non-smooth surface, wherein the at least one non-smooth vibrating surface is configured to contact and debride tissue on the wound surface and to enhance wound healing; and
at least one channel in fluid communication with an area adjacent the wound surface for transfer of fluid to or from the wound surface.

22. A method for debriding wound tissue and promoting wound healing using the end-effector of claim 1, comprising the steps of:
providing ultrasound waves to a non-smooth surface configured for debriding wound tissue and promoting wound healing;
propagating the ultrasound waves to cause vibration of the non-smooth surface; and
contacting the vibrating surface configured for debriding with the wound tissue to debride the wound tissue and to promote wound healing.

23. The method according to claim 22, further comprising providing fluid from the end-effector to the wound tissue to irrigate the wound tissue.

24. The method according to claim 22, further comprising providing fluid from the end-effector to the wound tissue, wherein said fluid penetrates the wound tissue.

25. The method according to claim 24, wherein the fluid includes a drug.

26. The method according to claim 22, further comprising the step of transferring material to or from the wound.

27. The end-effector in accordance with claim 20, wherein the at least one non-smooth surface includes at least one protrusion.

28. The end-effector in accordance with claim 27, wherein the at least one protrusion includes at least one tooth.

29. The end-effector in accordance with claim 20, wherein the at least one non-smooth surface includes a two-dimensional array of teeth.

30. The end-effector in accordance with claim 20, wherein the at least one non-smooth surface includes a two-dimensional grid of teeth.

31. The end-effector in accordance with claim 20, wherein the at least one non-smooth surface includes a plurality of teeth, and wherein each of said plurality of teeth has the same tooth configuration.

32. An end-effector configured and dimensioned for being connected to an ultrasound device, said end-effector comprising:
means for receiving ultrasound waves generated and propagated by the ultrasound device;
means for debriding tissue on a wound surface and enhancing wound healing, said means for debriding tissue and enhancing wound healing comprising:
at least one non-smooth surface; and
means for propagating the received ultrasound waves to the at least one non-smooth surface for vibrating the at least one non-smooth surface, wherein the at least one non-smooth vibrating surface is configured to contact and debride tissue on the wound surface to enhance wound healing; and
means for treating the tissue therapeutically by promoting penetration of a fluid below the surface of the wound with the received ultrasound waves.

33. The end-effector in accordance with claim 32, wherein the at least one non-smooth surface includes a contact area for contacting and debriding the wound surface, and wherein the contact area is arrayed in a radial or circular pattern.

34. The end-effector in accordance with claim 32, wherein the at least one non-smooth surface includes at least one protrusion including at least one tooth.

35. The end-effector in accordance with claim 32, wherein the at least one non-smooth surface includes a two-dimensional array of teeth.

36. The end-effector in accordance with claim 32, wherein the at least one non-smooth surface includes a two-dimensional grid of teeth.

37. The end-effector in accordance with claim 32, wherein the at least one non-smooth surface includes a plurality of teeth, and wherein each of said plurality of teeth has the same tooth configuration.

38. The end-effector in accordance with claim 32, wherein the means for treating the wound therapeutically includes a channel at least partially circumscribed with an indentation for causing at least a portion of the ultrasound waves to be configured as torsional or rotational waves.

39. The end-effector in accordance with claim 32, wherein the means for treating the wound therapeutically includes causing at least a portion of the at least one non-smooth surface to rotate.

40. The end-effector in accordance with claim 32, wherein the ultrasound waves have frequencies in the range from 20 kHz-100 kHz.

41. The end-effector in accordance with claim 32, wherein the ultrasound waves have amplitudes in the range from 5 to 120 microns.

42. The end-effector in accordance with claim 32, further comprising at least one channel in fluid communication with an area adjacent the wound surface for transfer of material to or from the wound surface.

43. The end-effector in accordance with claim 20, wherein the at least one channel is configured for irrigation.

44. The end-effector in accordance with claim 20, wherein the at least one channel is configured for suction.

45. The end-effector in accordance with claim 20, comprising separate channels for irrigation and suction.

46. A method for debriding wound tissue and promoting wound healing using the end-effector of claim 21, comprising the steps of:
providing ultrasound waves to a non-smooth surface configured for debriding wound tissue and promoting wound healing;
propagating the ultrasound waves to cause vibration of the non-smooth surface;
providing fluid from the end-effector to the wound tissue; and
contacting the vibrating surface configured for debriding with the wound tissue to debride the wound tissue and to promote wound healing.

47. The method according to claim 46, wherein fluid is provided to the wound tissue to irrigate the wound tissue.

48. The method according to claim 46, wherein fluid provided to the wound tissue penetrates the wound tissue.

49. The method in accordance with claim 46, wherein the at least one non-smooth surface includes a plurality of teeth.

50. The method in accordance with claim 46, wherein the at least one non-smooth surface includes a plurality of teeth, and wherein each of said plurality of teeth has the same tooth configuration.
